(19) United States
(12) Patent Application Publication
(10) Pub. No.: US 2002/0188279 A1
(43) Pub. Date: Dec. 12, 2002

(54) Vacuum flow visualization

(76) Inventors: Paula M. Waddell, Baltimore, MD (US); Jeffrey A. Taylor, Columbia, MD (US); Glenn L. Hawkes, Ellicott City, MD (US); Ronald Friedman, Owings Mills, MD (US); Thomas C. Jones, Columbia, MD (US)

Correspondence Address: ROGER M. RATHBUN, 13 MARGARITA COURT, HILTON HEAD ISLAND, SC, 29926, US

(21) Appl. No.: 09/876,617

(22) Filed: Jun. 7, 2001

(51) Int. Cl. A61M 1/00

(52) U.S. Cl. 604/540

(57) ABSTRACT

A vacuum system for applying vacuum to the body cavity of a patient where a vacuum flow indicator is provided that provides a visual display of the relative flow in a vacuum line to a medical patient. The vacuum indictor has indicia indicative of the no flow condition, a low flow condition, a high flow condition of flow through the vacuum system and has a space between the indicia of high flow and no flow so that the caregiver can visually get a quick and easy perception of the relative flow through the system. The vacuum flow indicator is not intended to be an accurate means of measuring that flow but only a guide that can aid the caregiver in determining and correcting a potential or real problem in the vacuum system.

### BACKGROUND

The present invention relates to a vacuum system that can be used to withdraw fluids from a patient in a medical environment, and, more particularly, to a vacuum system that has a device that allows the user to have a visual perception of the relative flow of fluid in order to have a general indication of the relative magnitude of the flow through the vacuum withdrawal system.

There are in use today, considerable uses of vacuum systems that carry out the withdrawal of fluids from a patient cavity as well as other medical uses in a hospital for such vacuum withdrawal systems. In general, after many surgical operations, there is a need to remove certain fluids from a patient and, to that end, most hospitals normally have a pipeline supply or a source of vacuum to the patient rooms so that there is a source of vacuum present on site for use with a patient. Thus, it is relatively convenient for the hospital to simply attach a vacuum regulator to that source of vacuum in the patient room and have a regulated source of vacuum that can be established by the caregiver at the particular level of vacuum that is desired to be applied to the patient. Additionally, of course, from time to time, it is necessary for that caregiver to change the level of the vacuum applied to the patient by resetting the regulator.

As other components of typical vacuum systems, there is provided a collection container that receives and collects the quantity of fluid from the patient and that container is connected to a cannula or catheter that is, in turn, actually introduced into the particular patient cavity from which the fluids are desired to be withdrawn.

One of the present problems in such vacuum systems, however, is in determining a general flow of the fluid in the vacuum system. In the use of such hospital vacuum systems, it is of interest for the caregiver to have some indication of the flow from the patient, however, it is not particularly necessary that the indication of such flow be accurate and actually measure the precise flow from the patient. The problem with obtaining an accurate flow with such systems is that with hospital vacuum systems, the conditions in the vacuum lines vary considerably, that is, the conditions in the vacuum flow lines themselves may range from full vacuum to atmospheric pressure while, on the other hand, flow meters for vacuum systems are normally calibrated at a specific pressure, or level of vacuum. Therefore, since the conditions in the hospital systems vary so widely, it simply is not possible to have an inexpensive flow measuring unit that can be used in the environment that will provide the user with an accurate, quantitative, indication of the flow in the vacuum line.

Thus, the caregiver simply carries out the use of the vacuum system on a patient and is dependent upon various sensory inputs to determine whether there if a flow in the vacuum line. For example, the caregiver may rely upon a visual observation of material passing along the flow tube that is withdrawing the fluids from the patient, or rely upon some audible sounds to determine that there is a flow in the vacuum line. Neither of such indicators provides a reliable indication as there may be no visible materials currently being withdrawn from the patient and the audible level may change from system to system and not provide any real, reliable information as to the flow in the system.

In addition, the use of an audible indicator requires the caregiver to remain at the site of the vacuum equipment for a period of time to tune out other sounds in the room and to really listen for the indication of flow of fluid from the patient. As a still further technique, the caregiver may look at the collection bottle itself to make sure that some material is being collected, however, again, the system may experience a flow where there is no material being collected at that period of time, and again, it is necessary for the caregiver to spend time accessing the quantity of material in the collection bottle in order to visually perceive the increase, or absence of increase, of that quantity of material.

As examples of flow devices in vacuum flow systems used with patients, there is, in U.S. Pat. No. 1,155,271, a very simple use of a fluttering wing that moves with the flow of fluid, however, the device is, at most only an indicator of the direction of the flow and provides little information as to a real relative determination of that flow, that is, the user would certainly not recognize whether there were a high, medium or low flow with any facility. In addition there is the use of a rotating paddle wheel type of device in U.S. Pat. No. 5,543,095, however, again, it would not be possible for the caregiver to gain a real indication by means of a quick glance at the device to glean any meaningful information with respect to the flow through that device.

Accordingly, there is clearly a need for some type of device that can provide useful, albeit not necessarily quantitatively accurate, information with respect to the flow within the hospital vacuum system to allow the caregiver to at least have some indication as to the relative flow in the vacuum system by sensing that there is no flow indicated by the flow indication device. For instance, with a flow indication device, the caregiver can block the line to immediately ascertain whether the vacuum system has been properly established and that there are no leaks in the system. The caregiver can also determine with a glance whether the system is operating and whether the system is currently removing liquid or gases since the flow of a gas naturally produces a higher flow in the vacuum system than if there is a liquid material being withdrawn from the patient. Thus, it would be advantageous to have present in a hospital vacuum system some device that would at least provide a relative qualitative indication of the flow in a hospital vacuum system even if the quantitative accuracy is not high.

### SUMMARY OF THE INVENTION

Accordingly, the present invention relates to a method and device used in a hospital vacuum system withdrawing fluids from a patient by providing a visual indication of the relative flow through that vacuum line removing those fluids from the patient. As used herein, the convention will be used that follows the flow in the vacuum line, that is, the source of vacuum will be a downstream location and the patient cavity at an upstream location and the terms upstream and downstream will be used with reference to the flow of fluid in the direction from the patient toward the source of vacuum.

Thus, in accordance with the present invention, a vacuum flow indicator, preferably a flow tube with a float is interposed in the vacuum line between the patient and the vacuum source, and preferably in a convenient location for the caregiver, that allows the caregiver to quickly access the relative flow in that vacuum, and to that end, it is preferable that the device has a visual indicator or indicia that provides a visual perception of a no flow, low flow, high flow and, of some intermediate flow that is at some level of flow between the indicated high and low flow conditions. That intermediate flow is readily perceived by a space, preferable linear, between the high and low flow indicia so that the caregiver can immediately see a relative level of the flow intermediate the high and low flow indicia. Also a no flow condition is important where that no flow condition is indicated if the float does not move off of its bottom stop. That is, the caregiver can get a quick visual indication from the visual vacuum flow indicator whether the flow in the vacuum system is at high, low or at some relative level therebetween or is blocked and there are no flow conditions.

As such, while the present device of this invention is not capable of actually providing an accurate, quantitative reading of any particular flow, the present device can quickly give a visual qualitative determination to the caregiver as to whether the flow in the vacuum line from the patient to the vacuum source is high, low at some relative point therebetween or is blocked (no flow conditions) and the caregiver can thus glean useful information for use in adjusting or otherwise attending to the administration of that vacuum or the maintenance of the equipment providing that vacuum.

In a preferred embodiment, the vacuum flow indicator is constructed to be contained within the same housing or enclosure as the normal vacuum regulator and vacuum gauge so that the caregiver can look to only one convenient location for all of the information provided with respect to the particular vacuum system being employed. The caregiver, can, therefore, have a visual perception of the vacuum flow in the vacuum regulator in addition to the actual level of the vacuum being applied to the patient so that the caregiver will know whether effective suction is being provided to that that patient and can visually recognize the differing degrees of vacuum level that may indicate a problem such as an occlusion in the vacuum line. As such, having perceived the problem at an early time, the caregiver can take the necessary steps promptly to correct the problem while minimizing the potential harm to the patient.

Additional features and advantages of the invention will become apparent to those skilled in the art upon consideration of the following detailed description of preferred embodiments exemplifying the best mode of carrying out the invention.

### BRIEF DESCRIPTION OF THE DRAWINGS

FIG. 1 is a front view of a vacuum control module constructed in accordance with the present invention;

FIG. 2 is a front view of a further vacuum control module of the present invention;

FIG. 3 is a schematic view of one embodiment of the invention;

FIG. 4 is a schematic view of further embodiment of the present invention; and

FIG. 5 is a schematic view of a still further embodiment of the present invention.

### DETAILED DESCRIPTION OF THE INVENTION

Turning now to FIG. 1, there is shown a front view of a vacuum control module 10 constructed in accordance with the present invention. In this Figure, the vacuum control module 10 comprises a housing 12 that may be a standard gauge formed metal or molded plastic enclosure and which contains the various components that combine to make up the vacuum system. In particular, there is an outlet, not shown in FIG. 1, that is adapted to be connected to a wall source of vacuum such as the normal wall vacuum line that is commonly located in hospital rooms and such vacuum outlet is normally provided in the rear of the vacuum control module 10.

An inlet 14 is also affixed to the enclosure 12 and receives the flow of fluid from a patient so that the patient actually sees the level of vacuum established by the vacuum control module 10 to control the flow of the fluids from the patient in removing the fluids from that patient. As is also a standard component of vacuum systems, there is also a vacuum gauge 16 that is affixed to the enclosure and thus is a part of the vacuum control module 10. The vacuum gauge 16 has a front face that enables the caregiver to visually perceive the level of the vacuum that is desired to be applied to the patient.

That level of vacuum to the patient is controlled, conventionally, by a regulator 18 that comprises an actuator and a knob that can be rotated by the caregiver to establish and change the level of the vacuum to the patient. The vacuum regulator 18 is also affixed to the enclosure 12 so as to be conveniently located for the user to activate the regulator 18 while observing the level of the vacuum by means of the vacuum gauge 16.

As a new component, however, the vacuum system includes a vacuum flow indicator 20 also affixed to the enclosure 12 and which provides a visual indication to the caregiver of the relative amount of flow in the vacuum line and thus an indication of flow from the patient. As can be seen, the vacuum flow indicator 20 has a visually perceptible range of values, and therefore, there is a no flow indicia 22 that would be located at the bottom of the vertically oriented embodiment of the vacuum flow indicator 20 of FIG. 1 as well as a high flow indicia 24 that would be located at the top of the vacuum flow indicator 20. There is also a finite space or range that is located between the no flow indicia 22 and the high flow indicia 24 so that the caregiver can quick visual indication of the relative flow, that is, where the flow is intermediate the no and high flow of the system. The no flow or zero flow condition is important as it provides an indication that the system has been set up properly and is the one point that the flow condition as displayed by the vacuum flow indicator 20 is assured to be accurate.

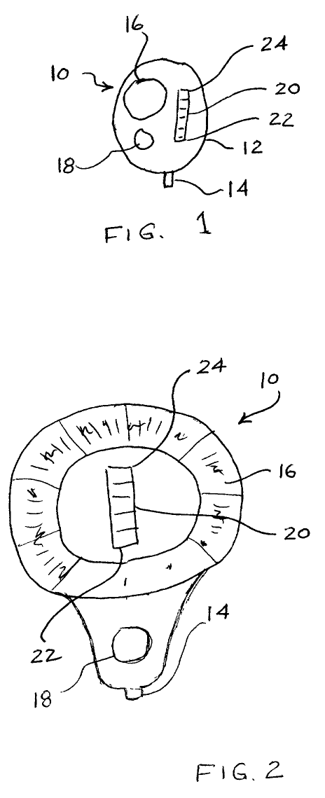

Turning briefly to FIG. 2, there is shown, a front view of an alternate embodiment to that of FIG. 1 and wherein the vacuum flow indicator 20 is located within the circular outer periphery of the indicia that are on the front face of the vacuum gauge 16 so that the caregiver can quickly and positively locate and visually read values for the level of vacuum by the vacuum gauge 16 as well as the flow in the vacuum line as visually perceived by means of the vacuum flow indicator 20.

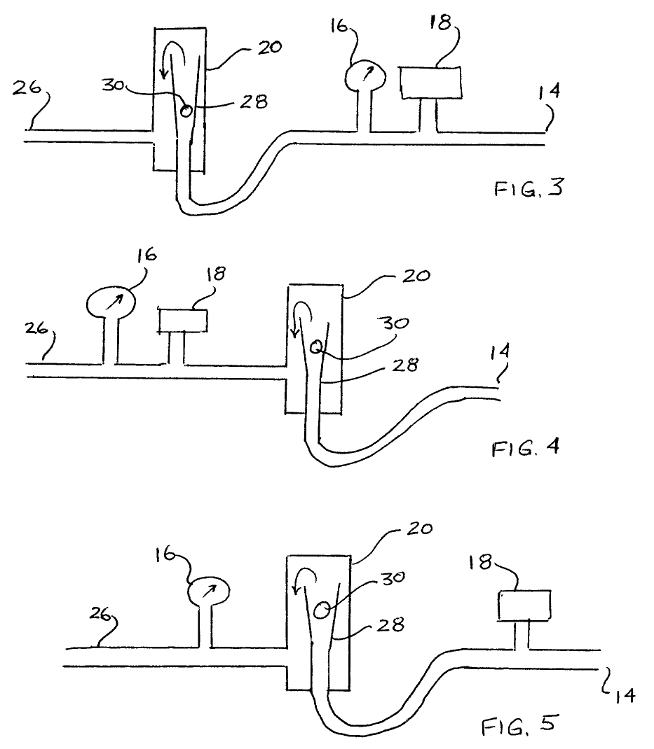

Next, in FIG. 3, there is a schematic view of a typical vacuum system that includes the vacuum flow indicator 20 of the present invention. In the FIG. 3 embodiment the vacuum flow indicator 20 is located between the outlet 26 that is adapted to be connected to the hospital wall supply of vacuum and the vacuum regulator 18 so that the flow displayed by the vacuum flow indicator 20 will be the flow downstream of the vacuum regulator 18. As also can be seen in FIG. 3, the vacuum flow indicator 20 is basically a conventional flow tube 28, commonly used with pressure flow systems, and which comprises a float 30 that is located within the flow tube 28 and is therefore indicative of the flow passing downstream of the vacuum regulator 18 to the wall vacuum system. The height of the float 30 indicates the flow therein through the flow tube 28 such that the higher the float 30 is located the higher the flow through that flow tube 28. The use of a flow tube device is well known with pressure flow systems and the technology for the flow tube in a vacuum system also with the purview of one skilled in that art.

In any event, turning now to FIG. 4, the is shown a further schematic view wherein the vacuum flow indicator 20 of the present invention is located upstream of the vacuum regulator 18 and the vacuum gauge 16 and thus will read the flow of the material from the patient to the vacuum flow regulator 18. Finally, in FIG. 5, there is shown a still further schematic where the vacuum flow indicator 20 is located between the vacuum regulator 18 and the vacuum gauge 16 and will indicate the flow intermediate the vacuum regulator 18 and the vacuum gauge 16. As can be seen by the FIG. 3-5 embodiments, it is not necessary that the vacuum flow indicator 20 be incorporated into or as a part of the housing containing the other vacuum controls as was the case with the FIG. 1 and 2 embodi8ments but can simply be an in line flow indicator. Though not the preferred embodiment, such an in line flow indicator could also provide the flow indication functions.

Thus, as can be seen, the vacuum flow indicator 20 can be positioned in a number of differing locations depending upon the flow desired to be monitored and the purpose of the vacuum flow indicator 20. In each instance, however, the vacuum flow indicator 20 provides a visual indication that can be easily and quickly perceived by the caregiver to obtain a relative flow, albeit not quantitatively accurate, by having a no flow indicia, a high flow indicia and some space between the indicia of no flow and high flow to enable the caregiver to perceive a general idea of the relative low in the desired area of the vacuum system.

Those skilled in the art will readily recognize numerous adaptations and modifications which can be made to the vacuum flow visualization system of the present invention which will result in an improved system yet all of which will fall within the scope and spirit of the present invention as defined in the following claims. Accordingly, the invention is to be limited only by the following claims and their equivalents.

We claim:

1. A vacuum system adapted to withdraw fluids from a body cavity of a patient, said vacuum system comprising a source of vacuum, a vacuum line providing communication between said source of vacuum and the patient cavity, a vacuum regulator in said vacuum line adapted to be adjusted to establish the level of vacuum to the patient cavity, a vacuum gauge to provide a visual indication of the level of vacuum in said vacuum line, and a flow indicator providing a visual indication of the flow through said vacuum line, said flow indicator having indicia visually indicating high flow and no flow and having a visual indication of a range of flows intermediate said indicia indicating said high flow and said no flow indicia.

2. A vacuum system as defined in claim 1 wherein said vacuum flow indicator comprises a flow tube and a float adapted to provide a visual indication of the flow therethrough.

3. A vacuum system as defined in claim 1 wherein said vacuum flow indicator is located intermediate said vacuum regulator and said vacuum gauge.

4. A vacuum system as defined in claim 1 wherein said vacuum flow indicator is located upstream of said vacuum regulator.

5. A vacuum system as defined in claim 1 wherein said vacuum flow indicator is located downstream of said vacuum regulator.

6. A vacuum control module for controlling vacuum to a patient, said vacuum control module comprising a housing, a vacuum regulator affixed to said housing and having a control means adapted to be activated by a user to set a level of vacuum to a patient, a vacuum gauge affixed to said housing an having a visual face displaying the level of vacuum through said vacuum regulator, and a flow indicator affixed to said housing, said flow indicator having visual indication of the relative flow of vacuum through said module, said flow indicator having indicia indicative of a high flow and a no flow and having a space between said indicia of high and no flow adapted to provide a visual indication of flows intermediate thereto.

7. A vacuum control module as defined in claim 6 wherein said visual face displaying said level of vacuum is generally circular and said vacuum flow indicator is located within said circular visual face.

8. A vacuum control module as defined in claim 7 wherein said vacuum flow indicator is a vertically oriented flow tube.

9. A vacuum control module as defined in claim 8 wherein said indicia displaying high flow is located at the top of said flow tube and said indicia displaying no flow is located at the bottom of said flow tube.

10. A vacuum control module as defined in claim 9 wherein space between said indicia displaying no and high flow is a finite linear distance.

11. A vacuum control module as defined in claim 6 wherein said vacuum flow indicator is a vertically oriented flow tube.

12. A vacuum control module as defined in claim 11 wherein said indicia displaying high flow is located at the top of said flow tube and said indicia displaying noflow is located at the bottom of said flow tube.
